(19) United States
(12) Patent Application Publication
(10) Pub. No.: US 2026/0217668 A1
(43) Pub. Date: Jul. 30, 2026

(54) CRYSTALLINE FORMS OF A LPA1 ANTAGONIST

(71) Applicant: Bristol-Myers Squibb Company, Princeton, NJ (US)

(72) Inventor: Shiwei TAO, Hillsborough, NJ (US)

(21) Appl. No.: 19/142,320

(22) PCT Filed: Dec. 22, 2023
(86) PCT No.: PCT/US2023/085811

§ 371 Date: Jun. 23, 2025

Related U.S. Application Data
(60) Provisional application No. 63/477,000, filed on Dec. 23, 2022.

(51) Int. Cl. C07D 401/04; A61K 31/4439

(52) U.S. Cl. CPC C07D 401/04; A61K 31/4439

(57) ABSTRACT

Described herein is the LPA₁ antagonist (1S,3S)-3-((2-methyl-6-(1-methyl-5-(((methyl(propyl)carbamoyl)oxy)methyl)-1H-1,2,3-triazol-4-yl)pyridin-3-yl)oxy)cyclohexane-1-carboxylic acid, including crystalline forms of Compound (A) and preparations thereof. Also disclosed are pharmaceutical compositions that include the LPA₁ antagonist, methods of using the LPA₁ antagonist for the treatment of interstitial lung disease.

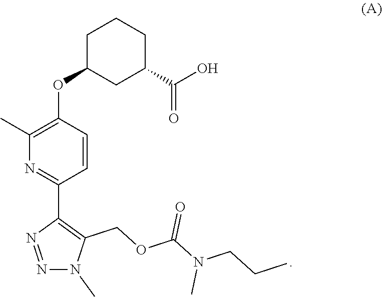 [SMILES: CCCN(C)C(=O)OCc1c(-c2ccc(O[C@H]3CCC[C@H](C(=O)O)C3)c(C)n2)nnn1C]

### CROSS-REFERENCE TO RELATED APPLICATIONS

This application claims the priority benefit of U.S. Provisional Application No. 63/477,000, filed Dec. 23, 2022, which is incorporated by reference herein in its entirety.

### TECHNICAL FIELD

Described herein is the LPA₁ antagonist (1S,3S)-3-((2-methyl-6-(1-methyl-5-(((methyl(propyl)carbamoyl)oxy)methyl)-1H-1,2,3-triazol-4-yl)pyridin-3-yl)oxy)cyclohexane-1-carboxylic acid, including crystalline forms. The disclosure also relates to preparations of crystalline forms, pharmaceutical compositions thereof, and methods of using the LPA₁ antagonist in the treatment of fibrotic diseases or conditions, including interstitial lung diseases.

### BACKGROUND

The interstitial lung diseases (ILDs) are a group of heterogeneous lung disorders classified together based on shared clinical features, parenchymal lung scarring (fibrosis) and/or inflammation with varying patterns of lung injury by imaging or histopathology. ILD may arise due to identifiable causes such as an underlying systemic autoimmune disease (e.g., systemic sclerosis or rheumatoid arthritis), environmental exposure (e.g., asbestos or silica), or result from medication toxicity, but is often idiopathic in nature. Idiopathic pulmonary fibrosis (IPF), one of the more common and most devastating types of ILD, is a chronic, progressive, and typically fatal lung disease of unknown cause characterized by worsening dyspnea, cough, loss of lung function due to scar formation in the lung and must have the pathological and radiographic pattern known as usual interstitial pneumonia (UIP) (Meltzer et al., Orphanet J. Rare Dis. 2008, 3:8). Beyond IPF, some patients with other forms of ILD develop a progressive fibrotic phenotype, characterized by worsening respiratory symptoms, lung function, progressive fibrosis on imaging and early mortality.

To date, two approved treatments, pirfenidone and nintedanib, significantly reduce the decline in lung function in patients with IPF and both appear to have modest effects on progression-free survival (Noble et al., Lancet 2011, 377 (9779), 1760-1769; King et al., N. Engl. J. Med. 2014, 370 (22), 2083-2092; Richeldi et al., New Engl J Med 2014, 370 (22), 2071-2082). However, many patients progress despite treatment. Another treatment option is lung transplantation, which has been shown to improve mortality in carefully selected patients, but not without complications (Kistler et al., BMC Pulmonary Med. 2014; 14:139). Despite these advances, there remains a large unmet need for a safe, well-tolerated and effective therapy for IPF that improves pulmonary function, delays disease progression, and reduces mortality.

Because of the clinical and pathophysiological similarities among IPF and other forms of progressive pulmonary fibrosis (PPF), it has been suggested that such disorders have a common pathobiologic mechanism regardless of the cause, with resultant progressive lung fibrosis, and thus could have a similar response to treatment as IPF (Raghu et al., Am. J. Respir. Crit. Care Med. 2022, 205, e18-e47; du Bois et al., Am. J. Respir. Crit. Care Med. 2012, 186, 712-715; Flaherty et al., N. Engl. J. Med. 2019, 381 (18), 1718-1727). Indeed, in the recently published INBUILD trial, patients with PPF with diverse etiologies were treated with nintedanib or placebo. Those treated with nintedanib had slower progression of lung fibrosis than those who received placebo, as demonstrated by a lower annual rate of decline in forced vital capacity (FVC) over the 52-week study period (Flaherty et al., N. Engl. J. Med. 2019, 381 (18), 1718-1727). The absolute treatment effects in the PPF study were similar in magnitude to those observed in the pivotal INPULSIS trials that led to approval of nintedanib for treatment of IPF. Furthermore, the IMPULSIS and INBUILD trials demonstrated that IPF and PPF patients were similar with respect to their rate of FVC decline. Based on data from the INBUILD trial, health authorities have also approved nintedanib for patients with PPF. Nonetheless, as many patients are unable to tolerate nintedanib due to gastrointestinal side effects, and because some patients may progress despite ongoing treatment with nintedanib, there remains an unmet need for well-tolerated, effective therapies for PPF (Flaherty et al., N. Engl. J. Med. 2019, 381 (18), 1718-1727).

Overall, there is a high unmet need for effective and tolerable treatments for patients with non-IPF, progressive pulmonary fibrosis (PPF) that exhibit disease progression. Fibrotic diseases such as these can be mediated by LPA, which signals via six LPA receptors (LPA₁₋₆). Signalling via LPA₁ appears to be fundamental in the pathogenesis of fibrotic diseases.

U.S. Patent Application Publication No. 2017/0360759 (PCT Application Publication No. WO2017/223016) discloses certain antagonists of lysophosphatidic acid (LPA) receptors for use in treating LPA-dependent or LPA-mediated conditions or diseases such as fibrosis of various organs, including the lung.

The compound of (1S,3S)-3-((2-methyl-6-(1-methyl-5-(((methyl(propyl) carbamoyl)oxy)methyl)-1H-1,2,3-triazol-4-yl)pyridin-3-yl)oxy)cyclohexane-1-carboxylic acid (hereafter referred as to “Compound A”) is described in U.S. Patent Application Publication No. 2017/0360759.

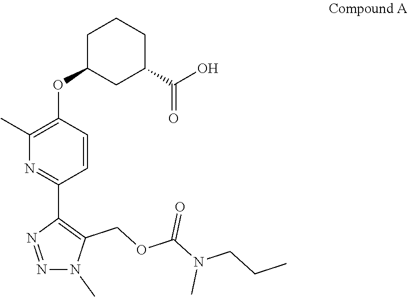 [SMILES: CCCN(C)C(=O)OCc1c(-c2ccc(O[C@H]3CCC[C@H](C(=O)O)C3)c(C)n2)nnn1C]

Compound A is a potent LPA₁ antagonist in vitro (LPA₁K_{b}=6.9 nM in CHO cells overexpressing human LPA₁ and LPA₁K_{b}=5.9 nM in normal human lung fibroblasts). Compound A is currently in clinical development as a therapy for IPF and PF-ILD. The present disclosure provides methods of treating interstitial lung disease using Compound A.

### BRIEF DESCRIPTION OF THE DRAWINGS

FIG. 1 shows a powder X-ray diffraction (PXRD) pattern of Form A.

FIG. 2 shows a differential scanning calorimetry (DSC) thermogram of Form A.

FIG. 3 shows a thermogravimetric analysis (TGA) thermogram of Form A.

FIG. 4 shows moisture sorption isotherms of Form A.

### SUMMARY

In some aspects, the present disclosure provides a crystalline Form A of Compound A:

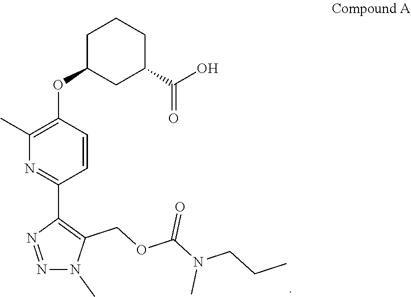 [SMILES: CCCN(C)C(=O)OCc1c(-c2ccc(O[C@H]3CCC[C@H](C(=O)O)C3)c(C)n2)nnn1C]

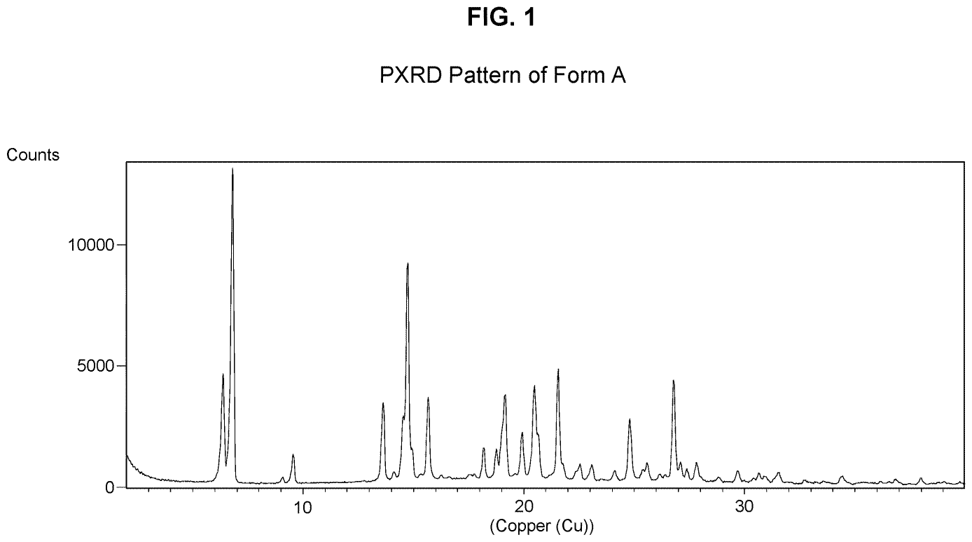

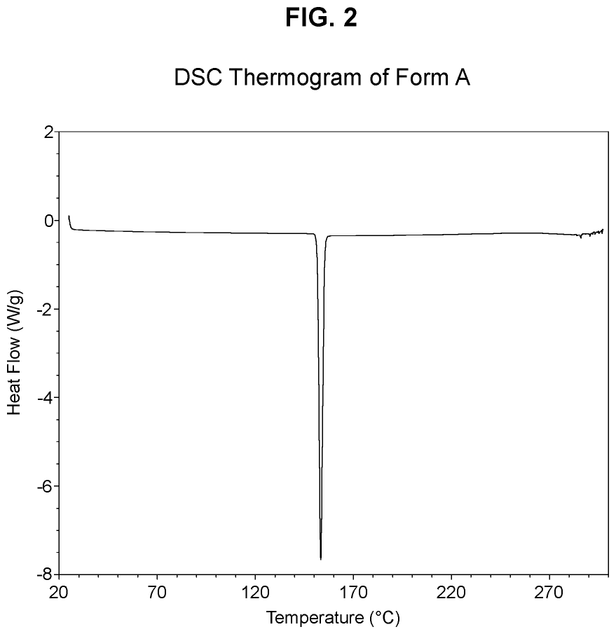

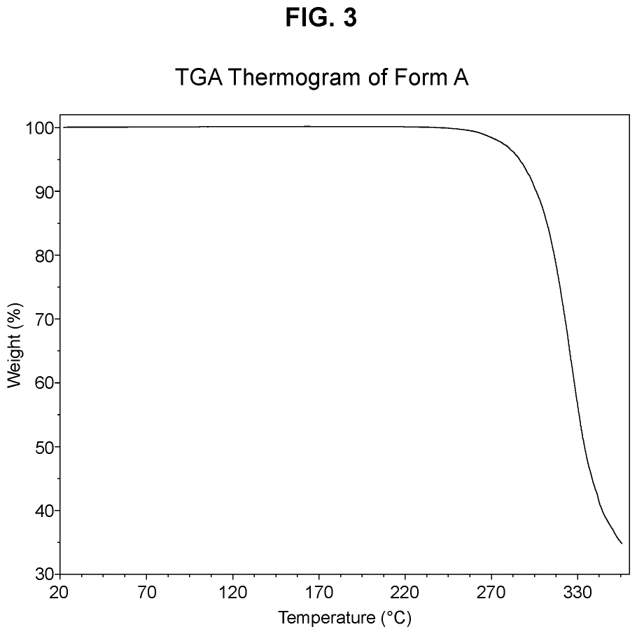

In some aspects, the crystal form is characterized by at least one of the following:
a) single crystal structure having unit cell parameters substantially equal to

| Crystal system, space group | Triclinic, P1 | |
|---|---|---|
| Unit cell dimensions | a = 6.53 ± 0.10 \acute{Å} | alpha = 92.8 ± 1.0° |
| | b = 13.06 ± 0.10 \acute{Å} | beta = 95.5 ± 1.0° |
| | c = 14.04 ± 0.10 \acute{Å} | gamma = 93.0 ± 1.0° |
| Volume | 1189(20) \acute{Å}³ | |
| Density (calculated) | 1.239 g/cm³ | |
| Temperature | room temperature | |

wherein measurement of the single crystal structure is at room temperature;
b) a powder x-ray diffraction pattern substantially the same as shown in FIG. 1;
c) a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å);
d) a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å);
e) a differential scanning calorimetry thermogram substantially similar to the one as shown in FIG. 2;
f) a differential scanning calorimetry thermogram with an endotherm having an onset at about 152° C.; and/or
g) a thermal gravimetric analysis thermogram substantially similar to the one as shown in FIG. 3.

In some aspects, the crystalline Form A has a single crystal structure having unit cell parameters substantially equal to

| Crystal system, space group | Triclinic, P1 | |
|---|---|---|
| Unit cell dimensions | a = 6.53 ± 0.10 \acute{Å} | alpha = 92.8 ± 1.0° |
| | b = 13.06 ± 0.10 \acute{Å} | beta = 95.5 ± 1.0° |
| | c = 14.04 ± 0.10 \acute{Å} | gamma = 93.0 ± 1.0° |
| Volume | 1189(20) \acute{Å}³ | |
| Density (calculated) | 1.239 g/cm³ | |
| Temperature | room temperature | |

wherein measurement of the single crystal structure is at room temperature.

In some aspects, crystalline Form A is characterized by a powder x-ray diffraction pattern substantially the same as shown in FIG. 1.

In some aspects, crystalline Form A is characterized by a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å).

In some aspects, Form A has a powder x-ray diffraction pattern comprising 2 or more peaks at 2θ values selected from 6.4±0.2, 6.8±0.2, 13.6±0.2, 15.7±0.2, and 21.6±0.2 (obtained at room temperature and CuKα λ=1.5418 Å).

In some aspects, Form A has a powder x-ray diffraction pattern comprising 3 or more peaks at 2θ values selected from 6.4±0.2, 6.8±0.2, 13.6±0.2, 15.7±0.2, and 21.6±0.2 (obtained at room temperature and CuKα λ=1.5418 Å).

In some aspects, crystalline Form A is characterized by a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å).

In some aspects, Form A has a powder x-ray diffraction pattern comprising 4 or more peaks at 26 values selected from 6.4±0.2, 6.8±0.2, 13.6±0.2, 15.7±0.2, and 21.6±0.2 (obtained at room temperature and CuKα λ=1.5418 Å). In some aspects, crystalline Form A is characterized by a differential scanning calorimetry thermogram substantially similar to the one as shown in FIG. 2.

In some aspects, crystalline Form A is characterized by a differential scanning calorimetry thermogram with an endotherm having an onset at about 152° C.

In some aspects, crystalline Form A is characterized by a thermal gravimetric analysis thermogram substantially similar to the one as shown in FIG. 3.

In some aspects, the crystalline form is in substantially pure form.

In some aspects, the present disclosure provides a pharmaceutical composition, which comprises a pharmaceutically acceptable carrier and the crystalline Form A of claims 1-10, alone or in combination with another therapeutic agent.

In some aspects, the crystal form described herein is used in treating interstitial lung disease. In some aspects, the interstitial lung disease is idiopathic pulmonary fibrosis (IPF). In some aspects, the interstitial lung disease is progressive pulmonary fibrosis (PPF).

### DETAILED DESCRIPTION

The present disclosure provides crystalline Compound A:

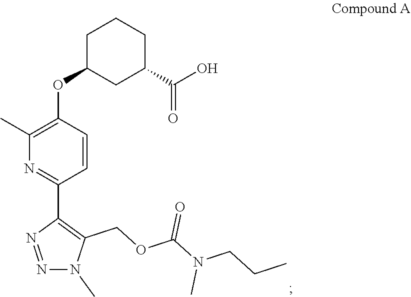 [SMILES: CCCN(C)C(=O)OCc1c(-c2ccc(O[C@H]3CCC[C@H](C(=O)O)C3)c(C)n2)nnn1C]

methods of preparing and using the crystalline compound thereof.

In order that the present description can be more readily understood, certain terms are first defined. Additional definitions are set forth throughout the detailed description.

### I. Definitions

Unless otherwise stated, the following terms used in this application, including the specification and claims, have the definitions given below. It must be noted that, as used in the specification and the appended claims, the singular forms “a,” “an,” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. Furthermore, use of the term “including” as well as other forms, such as “include,” “includes,” and “included,” is not limiting. The section headings used herein are for organizational purposes only and are not to be construed as limiting the subject matter described.

All measurements are subject to experimental error and are within the spirit of the invention.

The name used herein to characterize a specific form, e.g., “Form A”, should not be considered limiting with respect to any other substance possessing similar or identical physical and chemical characteristics, but rather it should be understood that the designation is a mere identifier that should be interpreted according to the characterization information also presented herein.

The term “about” as used in connection with a numerical value throughout the specification and the claims denotes an interval of accuracy, familiar and acceptable to a person skilled in the art. Such interval of accuracy is ±10%.

The term “acceptable” with respect to a formulation, composition or ingredient, as used herein, means having no persistent detrimental effect on the general health of the subject being treated.

The terms “administration of” and or “administering a” compound or composition should be understood to mean providing a compound or composition described herein to one or more subjects.

As used herein, “amorphous” refers to a solid form of a molecule, atom, and/or ions that is not crystalline. An amorphous solid does not display a definitive X-ray diffraction pattern.

The term “antagonist”, as used herein, refers to a molecule such as a compound, which diminishes, inhibits, or prevents the action of another molecule or the activity of a receptor site. Antagonists include, but are not limited to, competitive antagonists, non-competitive antagonists, uncompetitive antagonists, partial agonists and inverse agonists.

The terms “co-administration” or the like, as used herein, are meant to encompass administration of the selected therapeutic agents to a single subject, and are intended to include treatment regimens in which the agents are administered by the same or different route of administration or at the same or different time.

As used herein, the term “DSC” refers to differential scanning calorimetry. The term “TGA” refers to thermogravimetric analysis.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case can be determined using techniques, such as a dose escalation study.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “polymorphs” refer to crystalline forms having the same chemical structure but different spatial arrangements of the molecules and/or ions forming the crystals.

The term “room temperature” generally means approximately 22° C., but can vary up or down by up to 7° C.

The term “subject” encompasses mammals. Examples of mammals include, but are not limited to, humans, chimpanzees, apes, monkey, cattle, horses, sheep, goats, swine, rabbits, dogs, cats, rodents, rats, mice guinea pigs, and the like. In one embodiment, the mammal is a human.

As used herein, “substantially pure,” when used in reference to a crystal form, means a compound having a purity greater than 90 weight %, including greater than 90, 91, 92, 93, 94, 95, 96, 97, 98, and 99 weight %, and also including equal to about 100 weight % of the crystal form of Compound A, based on the weight of the compound. The remaining material comprises other form(s) of the compound, and/or reaction impurities and/or processing impurities arising from its preparation. For example, a crystal form of Compound A can be deemed substantially pure in that it has a purity greater than 90 weight %, as measured by means that are at this time known and generally accepted in the art, where the remaining less than 10 weight % of material comprises other form(s) of Compound A and/or reaction impurities and/or processing impurities.

When the term “substantially in accordance” is used in relation to PXRD, or XRPD patterns, it is to be understood that measurement of the peak locations for a given crystalline form of the same compound will vary within a margin of error. It is also to be understood that the intensities of the peaks can vary between different PXRD scans of the same crystalline form of the same compound. The relative intensities of the different peaks are not meant to be limiting to a comparison of different PXRD scans.

The terms “treat,” “treating,” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### II. Compound A

Compound A is described in U.S. Patent Application Publication No. 2017/0360759.

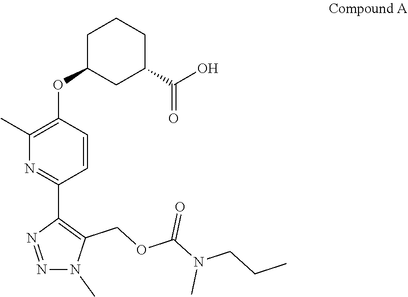 [SMILES: CCCN(C)C(=O)OCc1c(-c2ccc(O[C@H]3CCC[C@H](C(=O)O)C3)c(C)n2)nnn1C]

In preparing a pharmaceutical composition, a form of the active ingredient is sought that has a balance of desired properties, such as, for example, dissolution rate, solubility, bioavailability, and/or storage stability. For example, a form of the active ingredient is sought having sufficient solubility, bioavailability, and storage stability to prevent the sufficiently soluble and bioavailable form from converting, during the manufacture, preparation, and/or storage of the pharmaceutical composition, to another form having an undesirable solubility and/or bioavailability profile. In addition, a form of the active ingredient may also be sought that permits the active ingredient to be isolated and/or purified during, for example, a preparative process.

The present invention provides at least one form of Compound A that surprisingly affords a balance of properties sought in a pharmaceutical composition.

Described herein is Compound A including polymorphs and amorphous phases and methods of uses thereof, preferably an anhydrous Compound A.

In some aspects, the disclosure provides a crystalline anhydrous Compound A.

In some aspects, the disclosure provides a crystalline anhydrous form of Compound A, designated as Form A. In some embodiments, Compound A is provided as a crystalline material comprising Form A. In some embodiments, the crystalline Form A of Compound A is a neat crystalline form.

In some aspects, Compound A comprises crystalline form “Form A.” When dissolved, a crystalline form of Compound A loses its crystalline structure, and is therefore referred to as a solution of Compound A. All forms of the present invention, however, can be used for the preparation of liquid formulations in which the drug is dissolved or suspended. In addition, the crystalline Form A of Compound A can be incorporated into solid formulations.

As used herein, a PXRD (powder x-ray diffraction) or XRPD (x-ray powder diffraction) pattern “comprising” or having a number of peaks selected from a specified group of peaks, is intended to include PXRD patterns having additional peaks that are not included in the specified group of peaks. For example, a PXRD pattern comprising four or more peaks, preferably five or more, at 2θ values selected from: A, B, C, D, E, F, G, and H, is intended to include a PXRD pattern having: (a) four or more peaks, preferably five or more, at 2θ values selected from: A, B, C, D, E, F, G, and H; and (b) zero or more peaks that are not one of peaks A, B, C, D, E, F, G, and H.

In some aspects, Form A is characterized by a single crystal structure having unit cell parameters substantially equal to

| Crystal system, space group | Triclinic, P1 | |
|---|---|---|
| Unit cell dimensions | a = 6.53 ± 0.10 \acute{Å} | alpha = 92.8 ± 1.0° |
| | b = 13.06 ± 0.10 \acute{Å} | beta = 95.5 ± 1.0° |
| | c = 14.04 ± 0.10 \acute{Å} | gamma = 93.0 ± 1.0° |
| Volume | 1189(20) \acute{Å}³ | |
| Density (calculated) | 1.239 g/cm³ | |
| Temperature | room temperature | |

wherein measurement of the single crystal structure is at room temperature.

In some aspects, Form A has a powder x-ray diffraction pattern substantially the same as shown in FIG. 1.

In some aspects, Form A has a powder x-ray diffraction pattern comprising 2 or more peaks at 2θ values selected from 6.4±0.2, 6.8±0.2, 13.6±0.2, 15.7±0.2, and 21.6±0.2 (obtained at room temperature and CuKα λ=1.5418 Å). In some aspects, Form A has a powder x-ray diffraction pattern comprising 3 or more peaks at 2θ values selected from 6.4±0.2, 6.8±0.2, 13.6±0.2, 15.7±0.2, and 21.6±0.2 (obtained at room temperature and CuKα λ=1.5418 Å). In some aspects, Form A has a powder x-ray diffraction pattern comprising 4 or more peaks at 2θ values selected from 6.4±0.2, 6.8±0.2, 13.6±0.2, 15.7±0.2, and 21.6±0.2 (obtained at room temperature and CuKα λ=1.5418 Å).

In some aspects, Form A has a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å). In some aspects, Form A has a powder x-ray diffraction comprising 3 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å). In some aspects, Form A has a powder x-ray diffraction comprising 4 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å).

In some aspects, Form A has a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å). In some aspects, Form A has a powder x-ray diffraction pattern comprising 4 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å). In some aspects, Form A has a powder x-ray diffraction pattern comprising 5 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å).

In some aspects, Form A has a differential scanning calorimetry thermogram substantially similar to the one as shown in FIG. 2.

In some aspects, Form A has a differential scanning calorimetry thermogram with an endotherm having an onset at about 152° C.

In some aspects, Form A has a thermal gravimetric analysis thermogram substantially similar to the one as shown in FIG. 3.

In some aspects, the crystalline Form A is characterized by at least one of the following:
a) single crystal structure having unit cell parameters substantially equal to

| Crystal system, space group | Triclinic, P1 | |
|---|---|---|
| Unit cell dimensions | a = 6.53 ± 0.10 \acute{Å} | alpha = 92.8 ± 1.0° |
| | b = 13.06 ± 0.10 \acute{Å} | beta = 95.5 ± 1.0° |
| | c = 14.04 ± 0.10 \acute{Å} | gamma = 93.0 ± 1.0° |
| Volume | 1189(20) \acute{Å}³ | |
| Density (calculated) | 1.239 g/cm³ | |
| Temperature | room temperature | |

wherein measurement of the single crystal structure is at room temperature;
b) a powder x-ray diffraction pattern substantially the same as shown in FIG. 1;
c) a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å);
d) a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å);
e) a differential scanning calorimetry thermogram substantially similar to the one as shown in FIG. 2;
f) a differential scanning calorimetry thermogram with an endotherm having an onset at about 152° C.; and/or
g) a thermal gravimetric analysis thermogram substantially similar to the one as shown in FIG. 3.

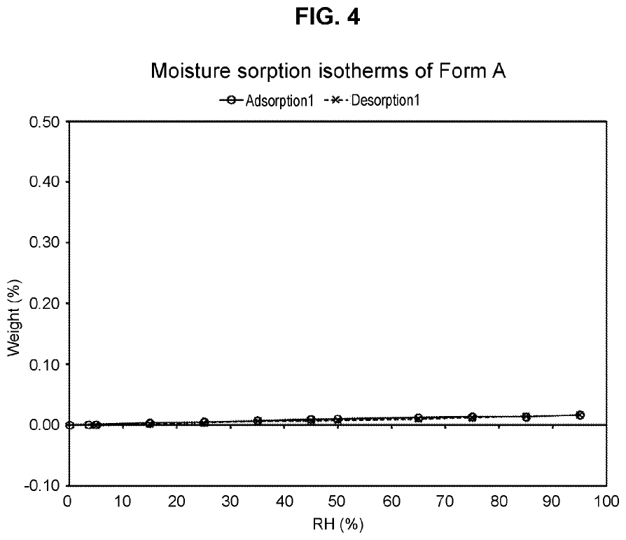

In some aspects, the crystalline Form A is characterized by at least one of the following:
a) single crystal structure having unit cell parameters substantially equal to

| Crystal system, space group | Triclinic, P1 | |
|---|---|---|
| Unit cell dimensions | a = 6.53 ± 0.10 \acute{Å} | alpha = 92.8 ± 1.0° |
| | b = 13.06 ± 0.10 \acute{Å} | beta = 95.5 ± 1.0° |
| | c = 14.04 ± 0.10 \acute{Å} | gamma = 93.0 ± 1.0° |
| Volume | 1189(20) \acute{Å}³ | |
| Density (calculated) | 1.239 g/cm | |
| Temperature | room temperature | |

wherein measurement of the single crystal structure is at room temperature;
b) a powder x-ray diffraction pattern substantially the same as shown in FIG. 1;
c) a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å);
d) a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å);
e) a differential scanning calorimetry thermogram substantially similar to the one as shown in FIG. 2;
f) a differential scanning calorimetry thermogram with an endotherm having an onset at about 152° C.; and/or
g) a thermal gravimetric analysis thermogram substantially similar to the one as shown in FIG. 3.
h) moisture sorption isotherms substantially similar to the ones as shown in FIG. 4; and/or
i) non-hygroscopicity.

In some aspects, the crystalline Form A is characterized by moisture sorption isotherms substantially as shown in FIG. 4.

In some aspects, the present invention describes a pharmaceutical composition comprising a therapeutically effective amount of the crystalline Form A of Compound A and a pharmaceutically acceptable carrier.

In some aspects, the crystalline Form A of Compound A is substantially pure. In some aspects, the crystalline Compound A contains at least about 90 wt. %, preferably at least about 95 wt. %, and more preferably at least about 99 wt. % Form A, based on weight of the crystalline Form A of Compound A.

In some embodiments, the crystalline Form A was obtained from tetrahydrofuran (TIF).

In some embodiments, the crystalline Form A was obtained from dichloromethane (DCM).

In some embodiments, the crystalline Form A was obtained from tetrahydrofuran (THF)/water.

In some embodiments, the crystalline Form A was obtained from 2-methyl THF/heptane.

In some embodiments, the crystalline Form A was obtained by dissolving in tert-Amyl alcohol (t-AmOH) followed by adding DCM and water, separating and concentrating the DCM layer followed by adding ethyl acetate (EtOAc) to the concentrated DCM layer, heating until complete dissolution, cooling, aging the resulting slurry, filtering, washing the wet cake with EtOAc, and drying under vacuum.

In some embodiments, the crystalline Form A was obtained by dissolving in tert-Amyl alcohol (t-AmOH), followed by charging with 2-propanol (IPA) and concentrating under vacuum, repeating charging/concentrating cycles, charging with water and heating the solution then cooling, followed by seeding with Form A seeds followed adding water, aging the resulting slurry, cooling, aging the slurry, filtering, washing the wet cake with a mixture of water:IPA:t-AmOH and drying under vacuum.

In some embodiments, the crystalline Form A is anhydrous.

In some embodiments, a crystalline Compound A, preferably Form A, is administered to a human.

In some embodiments, a crystalline Compound A, preferably Form A, is orally administered.

The present invention includes the use of the crystalline Compound A, preferably Form A, for use in therapy, for use in the formulation of a medicament for the treatment of ILDs.

### III. Compositions

In some aspects, the present disclosure provides compositions comprising Compound A. In some aspects, the present disclosure provides compositions comprising a crystal form of Compound A. In some aspects, the present disclosure provides compositions comprising Form A of Compound A. The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or ignore of the ingredient. Accordingly, the pharmaceutical compositions of the present invention encompass any composition made by mixing a compound of the present invention and a pharmaceutically acceptable carrier. By “pharmaceutically acceptable carrier” it is meant the carrier, diluent or excipient is compatible with the other ingredients of the formulation and not deleterious to the recipient thereof.

In some aspects, the compositions of the disclosure are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the compound of the disclosure can be in the form of a capsule. In some aspects, capsules can be immediate release capsules.

The compositions of the disclosure can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the compound of the disclosure can be in the form of a tablet. In some aspects, the compound of the disclosure can be in the form of a compressed tablet. In some aspects, the compound of the disclosure can be in the form of a film-coated compressed tablet. In some aspects, the compositions of the disclosure can be in the form of film-coated compressed tablets.

In some aspects, the compositions of the disclosure can be prepared by fluid bed granulation of the compound of the disclosure with one or more pharmaceutically acceptable carriers, vehicles, and/or excipients. In some aspects, the compositions of the disclosure can be prepared by fluid bed granulation process and can provide a tablet formulation with good flowability, good compressibility, fast dissolution, good stability, and/or minimal to no cracking. In some aspects, the fluid bed granulation process can allow preparation of formulations having high drug loading, such as over 70% or over 75% of a compound of the disclosure.

In some aspects, the compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, tsoft gelatin shells can contain a preservative to prevent the growth of microorganisms. Suitable preservatives include, but are not limited to, those as described herein, including methyl- and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. The capsules can also be coated as known by those of skill in the art in order to modify or sustain dissolution of the active ingredient.

In some aspects, the compositions of the disclosure can be in liquid or semisolid dosage forms, including emulsions, solutions, suspensions, elixirs, and syrups. In some aspects, the emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

In some aspects, the compositions of the disclosure for oral administration can be also provided in the forms of liposomes, micelles, microspheres, or nanosystems.

In some aspects, the compositions of the disclosure can be provided as non-effervescent or effervescent, granules and powders, to be reconstituted into a liquid dosage form. Pharmaceutically acceptable carriers and excipients used in the non-effervescent granules or powders can include, but are not limited to, diluents, sweeteners, wetting agents, and mixtures thereof. Pharmaceutically acceptable carriers and excipients used in the effervescent granules or powders can include, but are not limited to, organic acids, a source of carbon dioxide, and mixtures thereof.

Coloring and flavoring agents can be used in all of the above dosage forms. In addition, flavoring and sweetening agents can be especially useful in the formation of chewable tablets and lozenges.

In certain aspects, the compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

In certain aspects, Compound A, or the pharmaceutically acceptable salt and/or solvate thereof, can be administered orally. In some aspects, Compound A, or the pharmaceutically acceptable salt and/or solvate thereof, can be administered in a capsule. In some aspects, Compound A, or the pharmaceutically acceptable salt and/or solvate thereof, can be administered in a tablet.

Compound A is typically administered in an admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, sucrose, dextrose, dextrates, glucose, maltodextrin, mannitol, xylitol, sorbitol, cyclodextrins, calcium phosphate, calcium sulfate, starches, modified starches, methyl cellulose, microcrystalline cellulose, microcellulose, talc and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, glidants, flavoring agents, and coloring agents can also be incorporated into the mixture.

In still other aspects, using standard coating procedures, such as those described in Remington's Pharmaceutical Sciences, 20th Edition (2000), a film coating is provided around the formulation of Compound A.

Dosage forms (pharmaceutical compositions) suitable for administration can contain from about 1 milligram to about 300 milligrams of active ingredient per dosage unit. In these pharmaceutical compositions the active ingredient will ordinarily be present in an amount of about 0.5-95% by weight based on the total weight of the composition. In some aspects, dosage forms suitable for administration can contain from about 10 to about 240 milligrams of active ingredient per dosage unit. In some aspects, dosage forms suitable for administration can contain from about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, or about 240 mg of active ingredient per dosage unit.

In some aspects, the present disclosure provides pharmaceutical compositions which comprise Compound A as described herein, and at least one pharmaceutical acceptable carrier.

In some aspects, the present disclosure provides a pharmaceutical formulation for oral administration comprising:
(a) about 5 wt % to 40 wt % of Compound A;
(b) about 30 wt % to about 90 wt % of a diluent or a mixture of diluents;
(c) about 0 wt % to about 2 wt % of a glidant;
(d) about 2 wt % to about 10 wt % of a disintegrating agent; and
(e) about 0.25 wt % to about 4 wt % of a lubricant.

In some aspects, the Compound A of the pharmaceutical formulation comprises crystalline Form A. In some aspects the pharmaceutical formulation for oral administration is a tablet.

In some aspects, the present disclosure provides a pharmaceutical formulation for oral administration comprising:
(a) about 10 wt % to 30 wt % of Compound A;
(b) about 40 wt % to about 85 wt % of a diluent or a mixture of diluents;
(c) about 0 wt % to about 2 wt % of a glidant;
(d) about 2 wt % to about 10 wt % of a disintegrating agent; and
(e) about 0.25 wt % to about 4 wt % of a lubricant.

In some aspects, the Compound A of the pharmaceutical formulation comprises crystalline Form A. In some aspects the pharmaceutical formulation for oral administration is a tablet.

In some aspects, the diluents described herein are selected from lactose, sucrose, dextrose, dextrates, glucose, maltodextrin, mannitol, xylitol, sorbitol, cyclodextrins, calcium phosphate, calcium sulfate, starches, modified starches, methyl cellulose, microcrystalline cellulose, microcellulose, talc, and combinations thereof. In some aspects, the diluent or diluent mixture is selected from microcrystalline cellulose and anhydrous lactose.

As used herein, the term “glidant” refers a substance that, when added to a powder, improves the flowability of the powder, such as by reducing inter-particle friction. In some aspects, the glidant described herein is selected from silicas, silicon dioxide, CAB-0-SILM-SP, AEROSIL, talc, starch, magnesium aluminum silicates, and combinations thereof. In some aspects, the glidant is silicon dioxide.

In some aspects, the disintegrating agent described herein is selected from natural starch, a pregelatinized starch, a sodium starch, methyl crystalline cellulose, methylcellulose, croscarmellose, croscarmellose sodium, cross-linked sodium carboxymethylcellulose, cross-linked carboxymethylcellulose, cross-linked croscarmellose, cross-linked starch such as sodium starch glycolate, cross-linked polymer such as crospovidone, cross-linked polyvinylpyrrolidone, sodium alginate, a clay, a gum, and combinations thereof. In some aspects, the disintegrating agent is croscarmellose sodium.

In some aspects, the surfactant described herein is selected from sodium lauryl sulfate, sorbitan monooleate, polyoxyethylene sorbitan monooleate, polysorbates, polaxomers, bile salts, glyceryl monostearate, copolymers of ethylene oxide, propylene oxide, and combinations thereof. In some aspects, the surfactant is sodium lauryl sulfate.

In some aspects, the lubricant described herein is selected from stearic acid, calcium hydroxide, talc, corn starch, sodium stearyl fumerate, stearic acid, sodium oleate, sodium stearate, sodium benzoate, sodium acetate, sodium chloride, magnesium stearate, zinc stearate, waxes, and combinations hereof. In some aspects, the lubricant is magnesium stearate.

In some aspects, provided herein is a pharmaceutical formulation for oral administration comprising:
(a) about 5 wt % to 40 wt % of Compound A;
(b) about 15 wt % to about 70 wt % of microcrystalline cellulose and about 15 wt % to about 70 wt % of anhydrous lactose;
(c) about 0 wt % to about 2 wt % of silicon dioxide;
(d) about 2 wt % to about 6 wt % of croscarmellose sodium; and
(e) about 0.25 wt % to about 1.5 wt % of magnesium stearate.

In some aspects, the Compound A of the pharmaceutical formulation comprises crystalline Form A. In some aspects the pharmaceutical formulation for oral administration is a tablet.

In some aspects, provided herein is a pharmaceutical formulation for oral administration comprising:
(a) about 10 wt % to 30 wt % of Compound A;
(b) about 25 wt % to about 70 wt % of microcrystalline cellulose and about 25 wt to about 70 wt % of anhydrous lactose;
(c) about 0 wt % to about 2 wt % of silicon dioxide;
(d) about 2 wt % to about 6 wt % of croscarmellose sodium; and
(e) about 0.25 wt % to about 1.5 wt % of magnesium stearate.

In some aspects, the Compound A of the pharmaceutical formulation comprises crystalline Form A. In some aspects the pharmaceutical formulation for oral administration is a tablet. In some aspects, tablets can be prepared with the components provided in Tables 1 and/or 2.

TABLE 1
Components of Tablet Formulation

| Ingredient | Range |
|---|---|
| Compound A | 5% to 40% |
| Microcrystalline cellulose | 15% to 70% |
| Anhydrous Lactose | 15% to 70% |
| Silicon dioxide | 0% to 2% |
| Croscarmellose sodium | 2% to 6% |
| Magnesium Stearate | 0.25% to 1.5% |
| Total | Tablet weight range: |
| | 100 mg to 850 mg |

TABLE 2
Components of Tablet Formulation

| Ingredient | Range |
|---|---|
| Compound A | 10% to 30% |
| Microcrystalline cellulose | 25% to 70% |
| Anhydrous Lactose | 25% to 70% |
| Silicon dioxide | 0% to 2% |
| Croscarmellose sodium | 2% to 6% |
| Magnesium Stearate | 0.25% to 1.5% |
| Total | Tablet weight range: |
| | 100 mg to 850 mg |

In some aspects, the pharmaceutical compositions for oral administration can be prespred by direct compression or granulation (dry, wet, or melt granulation).

In some embodiments, the pharmaceutical composition further includes additional therapeutic agent(s). In some embodiments, the pharmaceutical composition further comprises one or more additional anti-fibrotic agents selected from pirfenidone, nintedanib, thalidomide, carlumab, FG-3019, fresolimumab, interferon alpha, lecithinized superoxide dismutase, simtuzumab, tanzisertib, tralokinumab, hu3G9, AM-152, IFN-gamma-lb, IW-001, PRM-151, PXS-25, pentoxifylline/N-acetyl-cysteine, pentoxifylline/vitamin E, salbutamol sulfate, [Sar9,Met(O2)11]-Substance P, pentoxifylline, mercaptamine bitartrate, obeticholic acid, aramchol, GFT-505, eicosapentaenoic acid ethyl ester, metformin, metreleptin, muromonab-CD3, oltipraz, IMM-124-E, MK-4074, PX-102, RO-5093151.

### IV. Methods of Treatment

The present disclosure provides methods of treating interstitial lung disease by administering Compound A, an LPA₁ antagonist. Lysophospholipids are membrane-derived bioactive lipid mediators. Lysophospholipids include, but are not limited to, lysophosphatidic acid (1-acyl-2-hydroxy-sn-glycero-3-phosphate; LPA), sphingosine 1-phosphate (SiP), lysophosphatidylcholine (LPC), and sphingosylphosphorylcholine (SPC). Lysophospholipids affect fundamental cellular functions that include cellular proliferation, differentiation, survival, migration, adhesion, invasion, and morphogenesis. These functions influence many biological processes that include neurogenesis, angiogenesis, wound healing, immunity, and carcinogenesis.

LPA acts through sets of specific G protein-coupled receptors (GPCRs) in an autocrine and paracrine fashion. LPA binding to its cognate GPCRs (LPA₁, LPA₂, LPA₃, LPA₄, LPA₅, LPA₆) activates intracellular signaling pathways to produce a variety of biological responses.

Lysophospholipids, such as LPA, are quantitatively minor lipid species compared to their major phospholipid counterparts (e.g., phosphatidylcholine, phosphatidylethanolamine, and sphingomyelin). LPA has a role as a biological effector molecule, and has a diverse range of physiological actions such as, but not limited to, effects on blood pressure, platelet activation, and smooth muscle contraction, and a variety of cellular effects, which include cell growth, cell rounding, neurite retraction, and actin stress fiber formation and cell migration. The effects of LPA are predominantly receptor mediated.

Activation of the LPA receptors (LPA₁, LPA₂, LPA₃, LPA₄, LPA₅, LPA₆) with LPA mediates a range of downstream signaling cascades. These include, but are not limited to, mitogen-activated protein kinase (MAPK) activation, adenylyl cyclase (AC) inhibition/activation, phospholipase C (PLC) activation/Ca²⁺ mobilization, arachidonic acid release, Akt/PKB activation, and the activation of small GTPases, Rho, ROCK, Rac, and Ras. Other pathways that are affected by LPA receptor activation include, but are not limited to, cyclic adenosine monophosphate (cAMP), cell division cycle 42/GTP-binding protein (Cdc42), proto-oncogene serine/threonine-protein kinase Raf (c-RAF), proto-oncogene tyrosine-protein kinase Src (c-src), extracellular signal-regulated kinase (ERK), focal adhesion kinase (FAK), guanine nucleotide exchange factor (GEF), glycogen synthase kinase 3b (GSK3b), c-jun amino-terminal kinase (JNK), MEK, myosin light chain II (MLC II), nuclear factor kB (NF-kB), N-methyl-D-aspartate (NMDA) receptor activation, phosphatidylinositol 3-kinase (PI3K), protein kinase A (PKA), protein kinase C (PKC), ras-related C3 botulinum toxin substrate 1 (RAC1). The actual pathway and realized end point are dependent on a range of variables that include receptor usage, cell type, expression level of a receptor or signaling protein, and LPA concentration. Nearly all mammalian cells, tissues and organs co-express several LPA-receptor subtypes, which indicates that LPA receptors signal in a cooperative manner. LPA₁, LPA₂, and LPA₃ share high amino acid sequence similarity.

LPA is produced from activated platelets, activated adipocytes, neuronal cells, and other cell types. Serum LPA is produced by multiple enzymatic pathways that involve monoacylglycerol kinase, phospholipase A₁, secretory phospholipase A₂, and lysophospholipase D (lysoPLD), including autotaxin. Several enzymes are involved in LPA degradation: lysophospholipase, lipid phosphate phosphatase, and LPA acyl transferase such as endophilin. LPA concentrations in human serum are estimated to be 1-5 μM. Serum LPA is bound to albumin, low-density lipoproteins, or other proteins, which possibly protect LPA from rapid degradation. LPA molecular species with different acyl chain lengths and saturation are naturally occurring, including 1-palmitoyl (16:0), 1-palmitoleoyl (16:1), 1-stearoyl (18:0), 1-oleoyl (18:1), 1-linoleoyl (18:2), and 1-arachidonyl (20:4) LPA. Quantitatively minor alkyl LPA has biological activities similar to acyl LPA, and different LPA species activate LPA receptor subtypes with varied efficacies.

LPA₁ (previously called VZG-1/EDG-2/mrec1.3) couples with three types of G proteins, G_{i/o}, G_{q}, and G_{12/13}. Through activation of these G proteins, LPA induces a range of cellular responses through LPA₁ including but not limited to: cell proliferation, serum-response element (SRE) activation, mitogen-activated protein kinase (MAPK) activation, adenylyl cyclase (AC) inhibition, phospholipase C (PLC) activation, Ca²⁺ mobilization, Akt activation, and Rho activation.

Wide expression of LPA₁ is observed in adult mice, with clear presence in testis, brain, heart, lung, small intestine, stomach, spleen, thymus, and skeletal muscle. Similarly, human tissues also express LPA₁; it is present in brain, heart, lung, placenta, colon, small intestine, prostate, testis, ovary, pancreas, spleen, kidney, skeletal muscle, and thymus.

The term “LPA-dependent”, as used herein, refers to conditions or disorders that would not occur, or would not occur to the same extent, in the absence of LPA.

The term “LPA-mediated,” as used herein, refers to refers to conditions or disorders that might occur in the absence of LPA but can occur in the presence of LPA.

The terms “fibrosis” and “fibrotic disease,” as used herein, refer to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract, such as idiopathic pulmonary fibrosis, scleroderma, and chronic nephropathies.

Exemplary diseases, disorders, or conditions that involve fibrosis include, but are not limited to: lung diseases associated with fibrosis, e.g., idiopathic pulmonary fibrosis, pulmonary fibrosis secondary to systemic inflammatory disease such as rheumatoid arthritis, scleroderma, lupus, cryptogenic fibrosing alveolitis, radiation induced fibrosis, chronic obstructive pulmonary disease (COPD), chronic asthma, silicosis, asbestos induced pulmonary or pleural fibrosis, acute lung injury and acute respiratory distress (including bacterial pneumonia induced, trauma induced, viral pneumonia induced, ventilator induced, non-pulmonary sepsis induced, and aspiration induced); chronic nephropathies associated with injury/fibrosis (kidney fibrosis), e.g., glomerulonephritis secondary to systemic inflammatory diseases such as lupus and scleroderma, diabetes, glomerular nephritis, focal segmental glomerular sclerosis, IgA nephropathy, hypertension, allograft and Alport; gut fibrosis, e.g., scleroderma, and radiation induced gut fibrosis; liver fibrosis, e.g., cirrhosis, alcohol induced liver fibrosis, nonalcoholic steatohepatitis (NASH), biliary duct injury, primary biliary cirrhosis, infection or viral induced liver fibrosis (e.g., chronic HCV infection), and autoimmune hepatitis; head and neck fibrosis, e.g., radiation induced; corneal scarring, e.g., LASIK (laser-assisted in situ keratomileusis), corneal transplant, and trabeculectomy; hypertrophic scarring and keloids, e.g., burn induced or surgical; and other fibrotic diseases, e.g., sarcoidosis, scleroderma, spinal cord injury/fibrosis, myelofibrosis, vascular restenosis, atherosclerosis, arteriosclerosis, Wegener's granulomatosis, mixed connective tissue disease, and Peyronie's disease.

Other diseases, disorders, or conditions where LPA₁ receptors can be involved include atherosclerosis, thrombosis, heart disease, vasculitis, formation of scar tissue, restenosis, phlebitis, COPD (chronic obstructive pulmonary disease), pulmonary hypertension, pulmonary fibrosis, pulmonary inflammation, bowel adhesions, bladder fibrosis and cystitis, fibrosis of the nasal passages, sinusitis, inflammation mediated by neutrophils, and fibrosis mediated by fibroblasts, dermatological disorders including proliferative or inflammatory disorders of the skin such as, atopic dermatitis, bullous disorders, collagenosis, psoriasis, psoriatic lesions, dermatitis, contact dermatitis, eczema, rosacea, wound healing, scarring, hypertrophic scarring, keloids, Kawasaki Disease, rosacea, Sjögren-Larsson Syndrome, and urticaria, respiratory diseases including asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia, and inflammatory/immune disorders including psoriasis, rheumatoid arthritis, vasculitis, inflammatory bowel disease, dermatitis, osteoarthritis, asthma, inflammatory muscle disease, allergic rhinitis, vaginitis, interstitial cystitis, scleroderma, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjögren's syndrome, thyroiditis (e.g., Hashimoto's and autoimmune thyroiditis), myasthenia gravis, autoimmune hemolytic anemia, multiple sclerosis, cystic fibrosis, chronic relapsing hepatitis, primary biliary cirrhosis, allergic conjunctivitis and atopic dermatitis.

Currently there are two approved treatments for interstitial lung disease: nintedanib and perfenidone, though several compounds are currently in development. In certain aspects of the present disclosure, the subject being administered Compound A is receiving concomitant treatment with one or more therapies for interstitial lung disease. In some aspects, the one or more therapies is selected from nintedanib and perfenidone.

In certain aspects, the subject is administered about 100 mg to about 150 mg of Compound A per day. In some aspects, the subject is administered about 110 mg to about 130 mg of Compound A per day. In some aspects the subject is administered about 100, about 105, about 110, about 115, about 120, about 125, about 130, about 135, about 140, about 145, or about 150 mg of Compound A per day. In some aspects, the subject is administered about 120 mg of Compound A per day.

In some aspects, the subject is administered Compound A once daily. In some aspects, the subject is administered Compound A twice daily. In some aspects, the subject is administered Compound A three times daily. In some aspects, the subject is administered Compound A four times daily. In some aspects, the subject is administered Compound A five times daily.

In some aspects, the subject is administered 120 mg once daily. In some aspects, the subject is administered a dose of 60 mg of Compound A twice daily. In some aspects, the subject is administered a dose of 40 mg of Compound A three times per day. In some aspects, the subject is administered a dose of 30 mg of Compound A four times per day. In some aspects, the subject is administered a dose of 24 mg five times per day.

In some aspects, the subject is administered Compound A with food. In some aspects, the subject is administered Compound A without food.

In some aspects, the subject administered Compound A experiences a slower disease progression than an untreated subject. In some aspects, disease progression is measured by the subject's decrease in forced vital capacity (FVC). In some aspects, the subject treated with Compound A experiences a smaller decline in forced vital capacity (FVC) after a treatment period compared to an untreated subject. FVC is the amount of air that a subject is able to forcibly exhale from his/her lungs after taking the deepest breath they can. FVC is typically measured using spirometry testing, which involves placing a special mask over the subject's face and having the subject inhale and exhale as forcibly as they can, while the measurements are collected.

In some aspects, disease progression can be measured by the time it takes a subject to experience a disease progression event. In some aspects, the subject administered Compound A experiences a greater time to first disease progression event after a treatment period than an untreated subject. In some aspects, the first disease progression event is absolute percentage predicted forced vital capacity (ppFVC) of ≥10% from baseline. An absolute or relative decline in % predicted FVC≥10% is associated with mortality. In some aspects, the subject treated with Compound A experiences a greater time to absolute percentage predicted forced vital capacity (ppFVC) of ≥10% from baseline than an untreated subject.

In some aspects, the first disease progression event is acute exacerbation (e.g., sudden worsening) of his/her lung fibrosis. In some aspects, the subject treated with Compound A experiences a greater time to acute exacerbation of lung fibrosis than an untreated subject.

In some aspects, the first disease progression event is respiratory hospitalization. In some aspects, the subject treated with Compound A experiences a greater time to respiratory hospitalization than an untreated subject.

In some aspects, the first disease progression event is lung transplantation. In some aspects, the subject treated with Compound A experiences a greater time to lung transplantation than an untreated subject.

In some aspects, the first disease progression event is mortality. In some aspects, the subject treated with Compound A experiences a greater time to all-cause mortality than an untreated subject.

In some aspects, the subject experiences a greater time to first disease progression event after a treatment period than an untreated subject, wherein the first disease progression event is selected from:
absolute percentage predicted forced vital capacity (ppFVC) of ≥10% from baseline;
acute exacerbation of lung fibrosis;
respiratory hospitalization;
lung transplantation; and
all-cause mortality.

In some aspects, disease progression is measured by a change in score in the Living with Pulmonary Fibrosis (L-PF) questionnaire. The Living with Pulmonary Fibrosis (L-PF) questionnaire assesses symptoms and quality of life in patients with fibrosing interstitial lung diseases (ILDs). Its Dyspnoea and Cough domains, whose items' responses are based on a 24-hour recall, have scores ranging from 0 to 100, with higher scores indicating greater symptom severity (see, for example, Swigris J J, et al. BMJ Open Resp Res 2022; 9:e001167. doi:10.1136/bmjresp-2021-001167). In some aspects, the subject administered Compound A experiences a smaller increase in cough domain score as measured by the Living with Pulmonary Fibrosis (L-PF) questionnaire over a treatment period than an untreated subject. In some aspects, the subject experiences a smaller increase in dyspnea score as measured by the Living with Pulmonary Fibrosis (L-PF) questionnaire over a treatment period than an untreated subject.

### EXAMPLES

### Example 1: Preparation of Crystalline Form A of Compound A

Crystal forms can be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of co-crystal forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture.

For crystallization techniques that employ solvent, the choice of solvent or solvents is typically dependent upon one or more factors, such as solubility of the compound, crystallization technique, and vapor pressure of the solvent. Combinations of solvents can be employed, for example, the compound can be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which can be heated to promote dissolution. The term “slurry”, as used herein, means a saturated solution of the compound, which can also contain an additional amount of the compound to afford a heterogeneous mixture of the compound and a solvent at a given temperature.

Seed crystals can be added to any crystallization mixture to promote crystallization. Seeding can be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971, 26, 369-377. In general, seeds of small size are needed to control effectively the growth of crystals in the batch. Seed of small size can be generated by sieving, milling, or micronizing of large crystals, or by micro-crystallization of solutions. Care should be taken that milling or micronizing of crystals does not result in any change in crystallinity form the desired crystal form (i.e., change to amorphous or to another polymorph).

A cooled crystallization mixture can be filtered under vacuum, and the isolated solids can be washed with a suitable solvent, such as cold recrystallization solvent, and dried under a nitrogen purge to afford the desired crystalline form. The isolated solids can be analyzed by a suitable spectroscopic or analytical technique, such as solid state nuclear magnetic resonance, differential scanning calorimetry, x-ray powder diffraction, or the like, to assure formation of the preferred crystalline form of the product. The resulting crystalline form is typically produced in an amount of greater than about 70 weight % isolated yield, preferably greater than 90 weight % isolated yield, based on the weight of the compound originally employed in the crystallization procedure. The product can be comilled or passed through a mesh screen to delump the product, if necessary.

The presence of more than one polymorph in a sample can be determined by techniques such as powder x-ray diffraction (PXRD) or by Raman or IR spectroscopy solid state nuclear magnetic resonance spectroscopy. For example, the presence of extra peaks in the comparison of an experimentally measured PXRD pattern with a simulated PXRD pattern can indicate more than one polymorph in the sample. The simulated PXRD can be calculated from single crystal x-ray data. see Smith, D. K., “A FORTRAN Program for Calculating X-Ray Powder Diffraction Patterns,” Lawrence Radiation Laboratory, Livermore, California, UCRL-7196 (April 1963).

The crystalline form of Compound A according to the invention can be characterized using various techniques, the operation of which are well known to those of ordinary skill in the art. The forms can be characterized and distinguished using single crystal x-ray diffraction, which is based on unit cell measurements of a single crystal of form at a fixed analytical temperature. A detailed description of unit cells is provided in Stout & Jensen, X-Ray Structure Determination: A Practical Guide, Macmillan Co., New York (1968), Chapter 3, which is herein incorporated by reference. Alternatively, the unique arrangement of atoms in spatial relation within the crystalline lattice can be characterized according to the observed fractional atomic coordinates. Another means of characterizing the crystalline structure is by powder x-ray diffraction analysis in which the diffraction profile is compared to a simulated profile representing pure powder material, both run at the same analytical temperature, and measurements for the subject form characterized as a series of 2q values (usually four or more).

Other means of characterizing the form can be used, such as solid state nuclear magnetic resonance (SSNMR), differential scanning calorimetry, thermogravimetric analysis and FT-Raman and FT-IR. These techniques can also be used in combination to characterize the subject form. In addition to the techniques specifically described herein, the presence of a particular crystalline form can be determined by other suitable analytical methods.

### Example 1A

150 mg Compound A was dissolved in 1.5 mL tetrahydrofuran (THF) at 20° C. 0.5 mL of this solution was subjected to flash evaporation using a centrifugal evaporator to yield solids of Form A. 0.5 mL of the same solution was subjected to slow evaporation at 20° C. to yield solids of Form A.

### Example 1B

200 mg Compound A was dissolved in 1 mL dichloromethane (DCM) at 20° C. 0.5 mL of this solution was subjected to flash evaporation using a centrifugal evaporator to yield solids of Form A. 0.5 mL of the same solution was subjected to slow evaporation at 20° C. to yield solids of Form A.

### Example 1C

100 mg of Compound A was dissolved in 0.5 mL of THF at 50° C. and kept stirring at 20° C. 0.5 mL of water was added to the clear solution, which resulted in solids of Form A.

### Example 1D

100 mg of Compound A was dissolved in 1 mL of 2-methyl THE at 50° C. and kept stirring at 20° C. 1 mL of n-heptane was added to the clear solution, which resulted in solids of Form A.

### Example 1E

A solution of Compound A in tert-Amyl alcohol (t-AmOH) was concentrated to 4 L/kg under vacuum, then was charged with 15 L/kg DCM and 10 L/kg water. Layers were split and the DCM layer was concentrated to 4 L/kg under vacuum. The DCM layer was charged with 8-10 L/kg ethyl acetate (EtOAc) and then concentrated to 4 L/kg under vacuum. An additional 8-10 L/kg EtOAc was charged then concentrated to 4 L/kg under vacuum. 6-8 L/kg EtOAc was charged and warmed to 70-83° C. until complete dissolution. The resulting slurry was cooled to 0-10° C. over at least 2 hours, followed by aging for at least an additional 12 hours. The slurry was filtered. The wet cake was washed with 3-5 L/kg EtOAc and dried under vacuum at 55-60° C. to yield solids of Form A.

### Example 1F

A solution of Compound A in t-AmOH was concentrated to 4 L/kg at 55° C. under vacuum, was then charged with 5 L/kg 2-propanol (IPA) and concentrated to 4 L/kg at 55° C. under vacuum. This process was repeated two additional times with 2×5 L/kg IPA. The batch was cooled to 30° C., was then charged with 1.3 L/kg water and heated to 45-55° C. The resulting warm solution was polish filtered and cooled to 30° C. 1 wt % Form A seeds were charged, followed by 2 L/kg water. After at least 6 hours, an additional 8.7 L/kg water was charged. The resulting slurry was cooled to 20° C. over at least 30 minutes, and the slurry was aged at least 3 hours. The solids were filtered and the wet cake was sequentially washed with 3 L/kg water:IPA:t-AmOH mixture (11:3:1 by volume) and 3 L/kg water, and dried under vacuum at 50-60° C. to solids of Form A.

### Example 1G

To a solution of isopropyl (1S,3S)-3-((2-methyl-6-(1-methyl-5-(((methyl(propyl)carbamoyl)oxy) methyl)-1H-1,2,3-triazol-4-yl)pyridin-3-yl)oxy)cyclohexane-1-carboxylate (500 mg, 1.025 mmol) in 1:1 THF/MeOH (10 mL) was added aqueous LiOH (1.538 mL of a 2M solution, 3.08 mmol). The reaction mixture was stirred at 50 C for 1 h, then was cooled to RT, and organic volatiles were removed in vacuo. The concentrated solution was washed with EtOAc, then was acidified to pH ~6-7 (1N aq HCl). This aqueous layer was extracted with EtOAc several times. The combined organic extracts were washed with water, dried (MgSO₄) and the MgSO₄ was filtered off. The EtOAc solution was concentrated in vacuo to yield solids of Form A.

The analytical data for the crystalline Compound A described herein were obtained using the following procedures.

### Single Crystal

Single crystal X-ray data were collected using a Bruker X8 Kappa diffractometer equipped with an APEX II CCD detector and a MICROSTAR microfocus rotating anode X-ray generator of monochromatic Cu Kα radiation. The single crystal was at room temperature during data collection.

Indexing and processing of the measured intensity data were carried out with the APEX2 program suite (Bruker AXS, Inc., 5465 East Cheryl Parkway, Madison, WI 53711 USA). The final unit cell parameters were determined using the full data set. The structures were solved by direct methods and refined by full-matrix least-squares approach using the SHELXTL software package (G. M. Sheldrick, SHELXTL v6.14, Bruker AXS, Madison, WI USA.). Structure refinements involved minimization of the function defined by Σw(|Fₒ|−|F_{c}|)², where w is an appropriate weighting factor based on errors in the observed intensities, Fₒ is the structure factor based on measured reflections, and F_{c} is the structure factor based on calculated reflections. Agreement between the refined crystal structure model and the experimental X-ray diffraction data is assessed by using the residual factors R=Σ∥Fₒ|−|F_{c}∥/Σ|Fₒ| and wR=[Σw(|Fₒ|−|F_{c}|)²/Σw|Fₒ|]^{1/2}. Difference Fourier maps were examined at all stages of refinement. All non-hydrogen atoms were refined with anisotropic thermal displacement parameters. Hydrogen atoms were introduced using idealized geometry with isotropic temperature factors and included in structure factor calculations with fixed parameters.

### Powder X-Ray Diffraction (PXRD)

PXRD diffractogram was acquired on a Bruker D8 Advance system using Cu Kα (40 kV/40 mA) radiation and a step size of 0.03° 2q and LynxEye detector, over a 2θ range of 2-40°. Configuration on the incident beam side: Göebel mirror, mirror exit slit (0.2 mm), 2.5 deg Soller slits, beam knife. Configuration on the diffracted beam side: anti-scatter slit (8 mm) and 2.5 deg. Soller slits. Sample was mounted flat on zero-background Si wafers.

### Differential Scanning Calorimetry (DSC)

DSC was conducted with a TA Instruments Q2000 differential scanning calorimeter equipped with an autosampler and a refrigerated cooling system under 40 mL/min N₂ purge for the Q2000. DSC thermogram was obtained at 15° C./min in crimped Al pan.

### Thermal Gravimetric Analysis (TGA)

TGA thermograms were obtained with a TA Instruments Q500 thermogravimetric analyzer under 40 mL/min N₂ purge for balance and 60 mL/min for sample in Al pan. TGA thermogram was obtained at 15° C./min.

### Moisture Sorption Isotherms

Moisture sorption isotherms were collected in a TA Instrument VTI-SA+ Vapor Sorption Analyzer using approximately 270 mg of sample in a 250 μL ceramic pan. The sample was dried at 30° C. until the loss rate of 0.005 wt %/min was obtained for 10 minutes. The sample was tested at 25° C. and 4, 5, 15, 25, 35, 45, 50, 65, 75, 85, and 95% RH. Equilibration at each RH was reached when the rate of 0.01 wt %/min for 35 minutes was achieved or a maximum of 600 minutes.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

What is claimed is:

1. A crystalline Form A of Compound A:

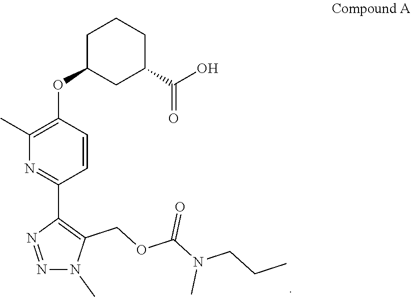 [SMILES: CCCN(C)C(=O)OCc1c(-c2ccc(O[C@H]3CCC[C@H](C(=O)O)C3)c(C)n2)nnn1C]

2. The crystalline Form A according to claim 1 is characterized by at least one of the following:
a) single crystal structure having unit cell parameters substantially equal to

| Crystal system, space group | Triclinic, P1 | |
|---|---|---|
| Unit cell dimensions | a = 6.53 ± 0.10 \acute{Å} | alpha = 92.8 ± 1.0° |
| | b = 13.06 ± 0.10 \acute{Å} | beta = 95.5 ± 1.0° |
| | c = 14.04 ± 0.10 \acute{Å} | gamma = 93.0 ± 1.0° |
| Volume | 1189(20) \acute{Å}³ | |
| Density (calculated) | 1.239 g/cm³ | |
| Temperature | room temperature | |

wherein measurement of the single crystal structure is at room temperature;
b) a powder x-ray diffraction pattern substantially the same as shown in FIG. 1;
c) a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å);
d) a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å);
e) a differential scanning calorimetry thermogram substantially similar to the one as shown in FIG. 2;
f) a differential scanning calorimetry thermogram with an endotherm having an onset at about 152° C.; and/or
g) a thermal gravimetric analysis thermogram substantially similar to the one as shown in FIG. 3.

3. The crystalline Form A of claim 1 or claim 2, wherein the crystalline Form A has a single crystal structure having unit cell parameters substantially equal to

| Crystal system, space group | Triclinic, P1 | |
|---|---|---|
| Unit cell dimensions | a = 6.53 ± 0.10 \acute{Å} | alpha = 92.8 ± 1.0° |
| | b = 13.06 ± 0.10 \acute{Å} | beta = 95.5 ± 1.0° |
| | c = 14.04 ± 0.10 \acute{Å} | gamma = 93.0 ± 1.0° |
| Volume | 1189(20) \acute{Å}³ | |
| Density (calculated) | 1.239 g/cm³ | |
| Temperature | room temperature | |

wherein measurement of the single crystal structure is at room temperature.

4. The crystalline Form A of claim 1 or claim 2, wherein the crystalline Form A is characterized by a powder x-ray diffraction pattern substantially the same as shown in FIG. 1.

5. The crystalline Form A of claim 1 or claim 2, wherein the crystalline Form A is characterized by a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKα λ=1.5418 Å).

6. The crystalline Form A of claim 1 or claim 2, wherein the crystalline Form A is characterized by a powder x-ray diffraction pattern comprising 2 or more peaks at 2θ values selected from 6.4±0.2, 6.8±0.2, 13.6±0.2, 15.7±0.2, and 21.6±0.2 (obtained at room temperature and CuKα λ=1.5418 Å)

7. The crystalline Form A of claim 1 or claim 2, wherein the crystalline Form A is characterized by a powder x-ray diffraction pattern comprising 3 or more peaks at 2θ values selected from 6.4±0.2, 6.8±0.2, 13.6±0.2, 15.7±0.2, and 21.6±0.2 (obtained at room temperature and CuKα λ=1.5418 Å).

8. The crystalline Form A of claim 1 or claim 2, wherein the crystalline Form A is characterized by a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKα λ=1.5418 Å).

9. The crystalline Form A of claim 1 or claim 2, wherein the crystalline Form A is characterized by a powder x-ray diffraction pattern comprising 4 or more peaks at 2θ values selected from 6.4±0.2, 6.8±0.2, 13.6±0.2, 15.7±0.2, and 21.6±0.2 (obtained at room temperature and CuKα λ=1.5418 Å).

10. The crystalline Form A of claim 1 or claim 2, wherein the crystalline Form A is characterized by a differential scanning calorimetry thermogram substantially similar to the one as shown in FIG. 2.

11. The crystalline Form A of claim 1 or claim 2, wherein the crystalline Form A is characterized by a differential scanning calorimetry thermogram with an endotherm having an onset at about 152° C.

12. The crystalline Form A of claim 1 or claim 2, wherein the crystalline Form A is characterized by a thermal gravimetric analysis thermogram substantially similar to the one as shown in FIG. 3.

13. The crystalline Form A of claims 1-12, in substantially pure form.

14. A pharmaceutical composition, which comprises a pharmaceutically acceptable carrier and the crystalline Form A of claims 1-13, alone or in combination with another therapeutic agent.

15. The crystalline Form A as defined in claims 1-13 and the pharmaceutical composition of claim 11 for use in treating interstitial lung disease.

16. The use of claim 15, wherein the interstitial lung disease is idiopathic pulmonary fibrosis (IPF).

17. The use of claim 15, wherein the interstitial lung disease is progressive pulmonary fibrosis (PPF).
